(19) United States
(12) Patent Application Publication
(10) Pub. No.: US 2025/0205300 A1
(43) Pub. Date: Jun. 26, 2025

(54) PLANT-BASED COMPOSITIONS AND METHODS FOR HORMONE SIGNALING, BLOOD GLUCOSE CONTROL, APPETITE CONTROL, AND WEIGHT MANAGEMENT

(71) Applicant: Alpine Biotech, LLC, Millcreek, UT (US)

(72) Inventor: Xuesheng Han, Salt Lake City, UT (US)

(73) Assignee: Alpine Biotech, LLC, Millcreek, UT (US)

(21) Appl. No.: 18/991,345

(22) Filed: Dec. 21, 2024

Related U.S. Application Data
(60) Provisional application No. 63/613,526, filed on Dec. 21, 2023.

(51) Int. Cl. A61K 36/82; A61K 31/202; A61K 31/4375; A61K 33/00; A61K 36/233; A61K 36/24; A61K 36/258; A61K 36/29; A61K 36/48; A61K 36/515; A61K 36/67; A61K 36/744; A61K 36/899; A61K 36/9066; A61K 45/06; A61P 3/04; A61P 3/08

(52) U.S. Cl. CPC A61K 36/82; A61K 31/202; A61K 31/4375; A61K 33/00; A61K 36/233; A61K 36/24; A61K 36/258; A61K 36/29; A61K 36/48; A61K 36/515; A61K 36/67; A61K 36/744; A61K 36/899; A61K 36/9066; A61K 45/06; A61P 3/04; A61P 3/08

(57) ABSTRACT

A composition for hormone signaling, blood glucose control, appetite control, and weight management is provided. The composition includes green tea leaf extract, gardenia fructus fruit powder, turmeric root extract, black pepper extract, fenugreek seed extract, ginseng root extract, and white kidney bean extract. A method for hormone signaling, blood glucose control, appetite control, and weight management is provided, comprising administering to the mammal the composition.

### CROSS-REFERENCE TO RELATED APPLICATION(S)

This application claims priority to and the benefit of U.S. Provisional Application Patent Ser. No. 63/613,526, filed on Dec. 21, 2023, the entire disclosure of which is hereby incorporated by reference.

### TECHNICAL FIELD

This disclosure relates to the technical field of hormone signaling, blood glucose control, appetite control, and weight management methods and compositions, in particular to plant-based compositions that modulate the endogenous secretion and activity of and sensitivity to glucagon-like peptide 1 (GLP-1) in animals and humans.

### BACKGROUND

Obesity epidemic is increasingly prevalent in industrialized nations. According to the World Health Organization, obesity is a condition in which excess body fat has accumulated to such an extent that health may be negatively affected. Excessive body weight is associated with various diseases, particularly cardiovascular diseases, diabetes mellitus type 2, obstructive sleep apnea, certain types of cancer, and osteoarthritis. As a result, obesity has been found to significantly reduce life expectancy.

Type 2 diabetes is associated with insulin resistance. Insulin is an important hormone that delivers glucose (sugar) to human cells. When a person is overweight or obese, the cells in the body more likely become less sensitive to the insulin that is released from the pancreas.

Glucagon-Like Peptide-1 Receptor Agonists (GLP-1RA), are a class of medications used in the treatment of type 2 diabetes and obesity. However, GLP-1RA drugs have limitations and undesirable side effects, such as gastrointestinal distress, hypoglycemia, pancreatitis, renal impairment, immunogenicity, thyroid cancers, et al.

Healthier and safer methods and compounds for blood glucose control, appetite control, and weight management are desirable.

### SUMMARY

Disclosed herein are implementations of hormone signaling, blood glucose control, appetite control, and weight management methods and compositions, in particular to plant-based compositions that modulate the endogenous secretion and activity of and sensitivity to GLP-1 in animals and humans.

One aspect of the disclosed implementations is a composition for modulating endogenous secretion and activity of and sensitivity to GLP-1. The composition may include: green tea leaf extract, gardenia fructus fruit powder, turmeric root extract, black pepper extract, fenugreek seed extract, ginseng root extract, and white kidney bean extract.

Another aspect of the disclosed implementations is a method for modulating the endogenous secretion and activity of and sensitivity to GLP-1.

These and other aspects of the present disclosure are disclosed in the following detailed description of the embodiments, the appended claims, and the accompanying figures.

### BRIEF DESCRIPTION OF THE DRAWINGS

The disclosure is best understood from the following detailed description when read in conjunction with the accompanying drawings. It is emphasized that, according to common practice, the various features of the drawings are not to scale. On the contrary, the dimensions of the various features are arbitrarily expanded or reduced for clarity.

FIG. 1 is a graph illustrating the effects of compositions in Human NCI-H716 cells according to an implementation.

FIG. 2 is a graph illustrating the effects of compositions in modulating plasma GLP-1 levels in the preclinical diabetes model according to an implementation.

FIG. 3 is a graph illustrating the anticipated effects of a composition in modulating fasting plasma GLP-1 levels, glucose levels, body weight, fat mass, muscle mass, and body mass index (BMI) in overweight and obese adult humans.

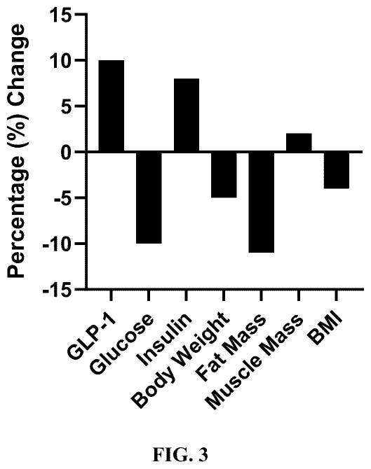

### DETAILED DESCRIPTION

The present invention relates to compositions and methods to enhance health and safety for hormone signaling, blood glucose control, appetite control, and weight management and the prevention or treatment of overweight or obesity. This invention also relates generally to the prevention and treatment of diseases and conditions mediated by glucagon-like peptide 1 (GLP-1) hormone and GLP-1 signaling. Specifically, the present invention relates to a novel composition of matter comprised of a mixture of compounds for use in the prevention and treatment of diseases and conditions mediated by GLP-1 hormone and GLP-1 signaling. In certain embodiments, the invention also relates to methods for improving glucose metabolism regulation to reduce the risk of developing insulin resistance and diabetes. The invention further provides dietary supplements for hormone signaling, blood glucose control, appetite control, and weight management.

This disclosure relates to plant-based compositions that modulate the endogenous secretion and activity of glucagon-like peptide 1 (GLP-1) and thus help hormone signaling, blood sugar glucose control, appetite control, and weight management in animals, and offer advantages compared to GLP-1RA drugs in the treatment of type 2 diabetes and obesity. For example, a plant-based composition of this disclosure may boost the endogenous secretion of GLP-1 in human L cells.

According to the present disclosure, a composition is provided to modulate endogenous secretion and activity of and sensitivity to GLP-1 in a mammal subject. The composition may include at least one plant-based compound that improves endogenous secretion and the activity and sensitivity to GLP-1.

In one implementation, the at least one plant-based compound may enable the mammal subject's cells to release GLP-1 more quickly, more efficiently, and/or more effectively than that would be without the at least one plant-based compound.

In one implementation, the at least one plant-based compound may improve the endogenous secretion and activity of and sensitivity to GLP-1, resulting in improved hormone signaling, blood sugar glucose control, appetite control, and weight management in the mammal subject.

In one implementation, a dose of the composition may include the at least one plant-based compound in a quantity that elicits a desired response, such as improved endogenous secretion and the activity of and sensitivity to GLP-1, resulting in improved hormone signaling, blood sugar glucose control, appetite control, and weight management in the mammal subject.

In one implementation, the composition comprises a mixture of green tea leaf extract, Gardeniae fructus extract, turmeric root extract, black pepper extract, fenugreek seed extract, ginseng root extract, and white kidney bean extract.

The green tea extract for use in the present composition may be selected from any suitable green tea leaf or green tea sources such as Sencha, Fukamushi Scncha, Gyokuro, Kabusecha, Matcha, Tencha, Genmaicha, Matcha, Shincha, Hojicha, Ichibanchagreen, Nibancha and Sanbancha tea, which are derived from the Camellia sinensis leaf. Green tea is abundant in polyphenols such as catechins. Examples of such catechins include epigallocatechin gallate, catechin, catechin gallate, epicatechin, gallocatechin, epigallocatechin, and epicatechin gallate. Preferably, the green tea extract for use in the composition comprises about 30% or more of epigallocatechin gallate by dry weight.

The green tea extract for use in the present composition may be either caffeinated or substantially decaffeinated, for example, having less than 1% caffeine by dry weight.

In one implementation, the green tea extract comprises about 1-50% of the dry weight of the composition.

In one implementation, the green tea extract comprises about 1-10% of the dry weight of the composition.

In one implementation, the green tea extract comprises about 10-50% of the dry weight of the composition.

The Gardeniae fructus extract for use in the present composition may be selected from any suitable Gardeniae sources, such as Zhizi (Gardenia jasminoides Ellis), Hainan Zhizi, and other species of the Gardenia genus.

In one implementation, the Gardeniae fructus extract comprises about 1-50% of the dry weight of the composition.

In one implementation, the Gardeniae fructus extract comprises about 1-10% of the dry weight of the composition.

In one implementation, the Gardeniae fructus extract comprises about 10-50% of the dry weight of the composition.

The turmeric root extract for use in the present composition may be obtained from any suitable source of the rhizomes of the Curcuma longa plants. Preferably, the turmeric root extract for use in the composition comprises about 95% or more of Curcumin.

In one implementation, the turmeric root extract comprises about 1-20% of the dry weight of the composition.

In one implementation, the turmeric root extract comprises about 1-4% of the dry weight of the composition.

In one implementation, the turmeric root extract comprises about 4-20% of the dry weight of the composition.

The black pepper extract for use in the present composition may be obtained from any suitable source of the berries of the Piper nigrum vine. Preferably, the black pepper extract for use in the composition comprises about 95% or more of piperine.

In one implementation, the black pepper extract comprises about 0.1-2% of the dry weight of the composition.

In one implementation, the black pepper extract comprises about 0.1-0.4% of the dry weight of the composition.

In one implementation, the black pepper extract comprises about 0.4-2% of the dry weight of the composition.

The Fenugreek seed extract for use in the present composition may be obtained from any suitable source of the Trigonella foemim-graecum plant seeds. Preferably, the Fenugreek seed extract for use in the composition comprises concentrated extract that is at least concentrated by a ratio of four parts of the raw material to one part of the final extract.

In one implementation, the Fenugreek seed extract comprises about 1-20% of the dry weight of the composition.

In one implementation, the Fenugreek seed extract comprises about 1-4% of the dry weight of the composition.

In one implementation, the Fenugreek seed extract comprises about 4-20% of the dry weight of the composition.

The ginseng root extract for use in the present composition may be selected from any suitable source of Panax ginseng plant roots. Preferably, the ginseng root extract for use in the composition comprises about 25% or more of ginsenosides.

In one implementation, the ginseng root extract comprises about 1-20% of the dry weight of the composition.

In one implementation, the ginseng root extract comprises about 1-4% of the dry weight of the composition.

In one implementation, the ginseng root extract comprises about 4-20% of the dry weight of the composition.

The white kidney bean extract for use in the present composition may be obtained from any suitable source of the Phaseolus vulgaris plant seeds. Preferably, the white kidney bean extract for use in the composition comprises concentrated extract that is at least concentrated by a ratio of four parts of the raw material to one part of the final extract.

In one implementation, the white kidney bean extract comprises about 1-20% of the dry weight of the composition.

In one implementation, the white kidney bean extract comprises about 1-4% of the dry weight of the composition.

In one implementation, the white kidney bean extract comprises about 4-20% of the dry weight of the composition.

In one implementation, the composition comprises a mixture of green tea leaf extract, Gardeniae fructus extract, turmeric root extract, black pepper extract, fenugreek seed extract, ginseng root extract, white kidney bean extract, berberine, and chromium.

In one implementation, the berberine is berberine chloride and comprises about 20-25% of the dry weight of the composition.

In one implementation, the chromium is trivalent chromium or chromic ion and comprises about 1-10% of the dry weight of the composition.

The weight management composition may be formulated for oral administration including, for example, solid, semi-solid, liquid, semi-liquid, powder, suspension, emulsion, solution, ready-to-drink beverage, gel, bar, pill, tablet or capsule form. The term “oral” or “orally” as used herein is intended to include any method in which the weight management composition is introduced into the digestive tract including the stomach and small intestine. Examples of oral administration may include administration via mouth, directly into the stomach using a feeding tube, through the nose to the stomach via a feeding tube and through the nose to the small intestine via a feeding tube. In a preferred embodiment, the weight management composition is provided as a loose powder, a bar or in capsules. The loose powdered composition may be reconstituted in water or any suitable liquid (such as juice, milk, saline, etc.) immediately prior to consumption. When provided in loose powdered form, the weight management composition may be packaged in individual use containers, packets or sachets, or in larger bulk containers. When provided as a powder in capsules, any suitable capsule may be used including gelatin and hard hydroxypropyl methylcellulose (also known as Hypromellose) capsules.

The present weight management composition is useful in a method to treat or improve at least one of the following in a mammal: body weight, body fat, endogenous secretion and the activity and sensitivity to GLP-1.

According to the present disclosure, a plant-based capsule is provided to modulate endogenous secretion and activity of and sensitivity to GLP-1 in a mammal subject. The plant-based capsule may include a composition includes at least one plant-based compound that improves endogenous secretion and the activity and sensitivity to GLP-1.

According to the present disclosure, a composition that consists of plant-based elements or ingredients is provided.

The processed plant product may comprise a processed portion of a plant that includes a sufficient amount of at least one compound that may modulate the endogenous secretion and activity of and sensitivity to GLP-1 and thus help hormone signaling, blood sugar glucose control, appetite control, and weight management in a subject (e.g., a mammal, etc.). Without limitation, the processed plant product may comprise a processed plant seed product. In a specific embodiment, the process plant product may comprise a byproduct of vegetable oil. In further embodiments, the processed plant product may comprise an extract of the processed portion of the plant or even a fraction of the extract.

The plant from which the processed plant product is obtained may comprise any plant that provides a suitable amount of at least one compound that may modulate the endogenous secretion and activity of and sensitivity to GLP-1 and thus help glucose and weight management in a subject (e.g., a mammal, etc.).

In another aspect, a method for modulating the endogenous secretion and activity of and sensitivity to GLP-1 and thus helping hormone signaling, blood sugar glucose control, appetite control, and weight management in a subject, such as a mammal (e.g. a human, a domesticated pet, etc.), is disclosed. The modulation of the endogenous secretion and activity of and sensitivity to GLP-1 and thus help hormone signaling, blood sugar glucose control, appetite control, and weight management include boosting the endogenous secretion and activity of GLP-1 in the subject, increasing the sensitivity to GLP-1, or the like.

Such a method may include administering to the subject a composition that includes at least one compound that may modulate the endogenous secretion and activity of and sensitivity to GLP-1 and thus hormone signaling, blood sugar glucose control, appetite control, and weight management in the subject. The composition that is administered to the subject may comprise a composition of this disclosure. The amount of the at least one compound in the composition (e.g., a dose of the composition, etc.) may be sufficient to the endogenous secretion and activity of and sensitivity to GLP-1 and thus hormone signaling, blood sugar glucose control, appetite control, and weight management in the subject (e.g., boost endogenous secretion of GLP-1, increase activity of GLP-1, increase sensitivity to GLP-1, lower glucose level, suppress appetite, lose weight, lose fat, etc.). For example, the amount of the at least one compound in the composition (e.g., a dose of the composition, etc.) may be sufficient to boost the endogenous secretion and activity of GLP-1 in a subject.

In one implementation, a mammal glucagon-like peptide 1 modulating composition includes: green tea leaf extract; gardenia fructus fruit powder; turmeric root extract; black pepper extract; fenugreek seed extract; ginseng root extract; and white kidney bean extract. The green tea leaf extract includes 30% or more of epigallocatechin gallate by dry weight; the turmeric root extract includes 95% or more of curcumin by dry weight; the black pepper extract includes 95% or more of piperine by dry weight; the ginseng root extract includes 25% or more of ginsenosides by dry weight; and the fenugreek seed extract and the white kidney bean extract were enriched in a 4:1 ratio.

In one implementation, the mammal glucagon-like peptide 1 modulating composition includes one or more selected from the group consisting of Citrus aurantium extract, Gentiana scabra extract, Bupleurum falcatum extract, Anemarrhena asphodeloides extract, Plasmodium falciparum extract, Sophorae flos extract, Alismatis rhizome extract, Nelumbinis folium extract, Cassiae semen extract, Poria cocos extract, Zea mays extract, α-linolenic acid, and Hoodia gordonii extract.

In one implementation, the mammal glucagon-like peptide 1 modulating composition includes one or more selected from the group consisting of α-linolenic acid, Bupleurum falcatum extract, Gentiana scabra extract, Hoodia gordonii extract, and Zea mays extract.

In one implementation, the mammal glucagon-like peptide 1 modulating composition includes chromium nicotinate.

In one implementation, the mammal glucagon-like peptide 1 modulating composition includes berberine, and the berberine is from Berberis Aristata bark extract.

In one implementation, the mammal glucagon-like peptide 1 modulating composition includes decaffeinated green tea leaf extract.

In one implementation, the mammal glucagon-like peptide 1 modulating composition includes the amount of each of the components corresponds to a ratio of: 0.1-1 green tea leaf extract; 0.1-1 gardenia fructus fruit powder; 0.04-1 turmeric root extract; 0.004-1 black pepper extract; 0.04-1 fenugreek seed extract; 0.04-1 ginseng root extract; and 0.04-1 white kidney bean extract.

In one implementation, the mammal glucagon-like peptide 1 modulating composition includes one or more selected from the group consisting of vitamin C, vitamin D, calcium, magnesium, phosphate, pyruvate, NADH, nitrates, and a mixture thereof.

In one implementation, the mammal glucagon-like peptide 1 modulating composition is administered in solid, semi-solid, liquid, semi-liquid, powder, suspension, emulsion, solution, ready-to-drink beverage, gel, bar, pill, tablet or capsule form.

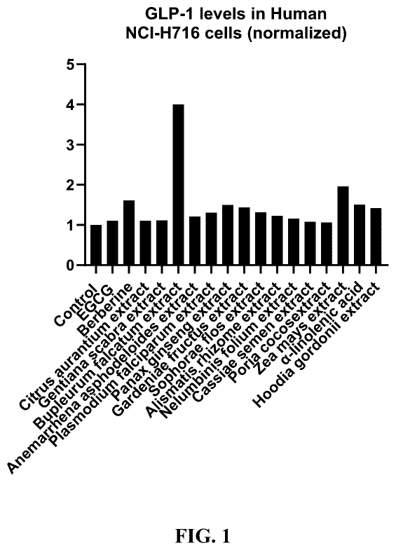

As illustrated in FIG. 1, in some implementations, epigallocatechin gallate (“EGCG”), Berberine, Citrus aurantium extract, Gentiana scabra extract Bupleurum falcatum extract, Anemarrhena asphodeloides extract, Plasmodium falciparum extract, Panax ginseng extract, Gardeniae fructus extract, Sophorae flos extract, Alismatis rhizome extract, Nelumbinis folium extract, Cassiae semen extract, Poria cocos extract, Zea mays extract, α-linolenic acid, and Hoodia gordonii extract modulate the secretion of GLP-1 to various degrees in Human NCI-H716 cells.

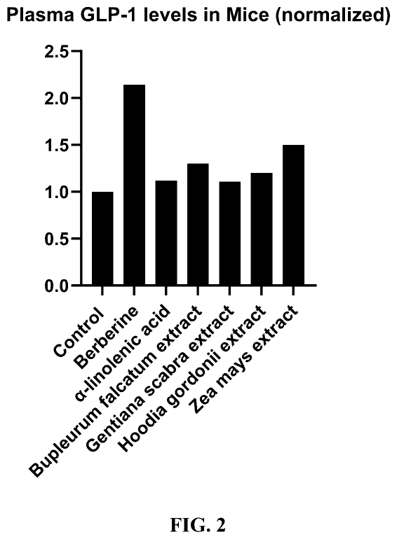

As illustrated in FIG. 2, in some implementations, Berberine, α-linolenic acid, Bupleurum falcatum extract, Gentiana scabra extract, Hoodia gordonii extract, Zea mays extract modulate plasmas GLP-1 levels in preclinical diabetes model.

### Example 1

GLP-1 modulating composition (“GB”) is designed to increase the endogenous production of GLP-1, improve the activity of GLP-1, enhance the body's response to GLP-1, and thus eventually help people reduce blood glucose, suppress food intake, and lose body fat. GB contains green tea (Camellia sinensis) leaf extract, Gardeniae (Gardenia jasminoides Ellis) fructus extract, Turmeric (Curcuma longa) root extract, Black pepper (Piper nigrum) extract, Fenugreek (Trigonella foemim-graecum) seed extract, Ginseng (Panax ginseng) root extract, and White kidney bean (Phaseolus vulgaris) extract. Each of these ingredients has been commonly consumed by humans as food sources or supplements and thus has a good safety profile.

To evaluate GB's potential as a safe and effective solution for glucose control and weight loss, the present study was designed to explore its effects on hormone signaling, blood sugar glucose control, appetite control, and weight management in relatively healthy yet overweight adult men and women over a period of 12 weeks.

### Materials and Methods

### Study Materials

Participants were instructed to take GB daily, right before their biggest meal of the day. GB contains green tea (Camellia sinensis) leaf extract, Gardeniae (Gardenia jasminoides Ellis) fructus extract, Turmeric (Curcuma longa) root extract, Black pepper (Piper nigrum) extract, Fenugreek (Trigonella foenum-graecum) seed extract, Ginseng (Panax ginseng) root extract, and White kidney bean (Phaseolus vulgaris) extract.

### Study Procedure

The study was reviewed and approved by an ethics committee before its commencement. The participants were recruited in the Salt Lake City area, Utah, USA. This study was registered on ClinicalTrials.gov (NCT06333496).

### Inclusion Criteria

Individuals who were at least 18 years old, non-smokers, and having a BMI between 25 and 29.9 were able to participate, if not currently taking a dietary supplement or medications for glucose control or weight loss. Regular exercise volunteers were asked to maintain their regimen consistently throughout the course of the 12-week study and caffeine drinking volunteers were asked to maintain their caffeine intake consistently throughout the course of the 12-week study.

### Exclusion Criteria

Participants were not allowed to participate if they were pregnant or planning to become pregnant in the following 12 weeks, or lactating. Individuals were not allowed to participate if they were taking any stimulant medications, or any other medications or supplements that might impact glucose control and weight loss.

### Participants Characteristics

Twelve (12) adult subjects were screened, ten (10) of them were enrolled and completed the study. Study participants were asked to maintain a consistent diet and exercise regimen throughout the study. At the beginning of the study, study participants were asked to fill out and sign an Information Form and Informed Consent.

Participants were supplied with one bottle of GB for each visit, labeled with their participant number. They were required to take the product consisting of 4 capsules per day, taken on an empty stomach right before their biggest meal of the day, everyday. Product was taken at the beginning of each visit for a total of 12 weeks and empty bottles were returned at each visit. Any dosing days that were missed were skipped and noted in the Questionnaire. Visits occurred at Week 0, Week 4, Week 8, and Week 12. Prior to each visit, participants were asked to fast and refrain from exercise for at least 4 hours.

### Measurements

Height was measured according to the US National Health and Nutrition Examination Survey III (NHANES III) protocol at Week 0 only. The following outcome measurements were measured at each visit.

Body weight, fat free mass, fat mass, body mass index (BMI), and basal metabolic rate were measured by a pre-calibrated InBody570 Body Composition Analyzer following manufacturer instructions (Cerritos, California). Body fat index (BFI) was calculated using body fat mass and height.

Waist hip ratio (WHR) was measured according to NHANES III protocol.

Blood pressure and heart rate were measured using the Omron Intellisense digital blood pressure monitor, Model HEM-739 according to the equipment manual (Vernon Hills, IL).

Questionnaires for positive/negative effects, energy levels, feelings of hunger, and satiety parameters. The presence of positive and negative effects was measured by a participant survey. Energy, hunger, and satiety were measured by Visual Analog Scale (VAS) incremented from 1 to 10.

Cholesterol, glucose, insulin, HbA1C and GLP-1 levels in blood were measured by certified third party labs. Blood samples were taken by a certified phlebotomist.

### Data Analysis

For the statistical analyses a series of linear mixed effects models were used. These are an extension of one-way analysis of variance that considers potential dependence within each subject. Week was used as the fixed effect, both as a numeric or linear term and as a categorical variable. Dunnet's post-hoc test was used to compare weeks 4, 8, and 12 to week 0 while controlling for the multiple comparisons. The response variables included Energy, Hungry, Fullness, Desire to Eat, Appetite, RMR, Systolic blood pressure, Diastolic blood pressure, Heart Rate, Total Cholesterol, Glucose, Body weight, BMI, Body fat, Fat mass, Fat free mass, BFI, and WHR. All analyses were performed using R version 4.4.1. A p value of ≤0.05 was considered statistically significant.

### Study Participants

Twelve (12) adult subjects were screened, and ten (10) participants (4 female and 6 male) completed the entire study, and thus were included for the data analysis. All participants were overweight (average BMI 26.95) at the beginning of the study. No adverse reaction was found or reported as illustrated in Table 1.

TABLE 1
Demographic Information of Study Participants
at Study Commencement.

| | |
|---|---|
| Age (years) (Mean, SD) | 35.80, 6.05 |
| Body Height (inches) (Mean, SD) | 66.68, 4.12 |
| Body weight (pounds) (Mean, SD) | 171.29, 24.27 |
| BMI (kg/m²) (Mean, SD) | 26.95, 1.25 |
| Race/Ethnicity | |
| White | 4 (40%) |
| Asian | 2 (20%) |
| Hispanic/Latino | 2 (20%) |
| African | 2 (20%) |
| Gender | |
| Female | 4 (40%) |
| Male | 6 (60%) |

### Subjective Parameters

Though study participants reported decreased Frequency of Exercise (Week 12 vs Week 0), they reported moderately increased Energy Level at Week 4, 8, and 12 compared to baseline Week 0, with Week 12 seeing 10.2% increase on average (p<0.05). Participants reported a significant decrease in Feeling of Hunger at Week 4, 8, and 12 compared to Week 0, specifically a 42.2% decrease on average at Week 12 (p<0.001). Participants also reported a significant increase in Feeling of Fullness at Week 4, 8, and 12 compared to Week 0, specifically an 80.5% increase on average at Week 12 (p<0.001). When asked about their Desire to Eat and Eat How Much Food, participants further reported significant decreases in both: specifically, a 48.6% decrease on Desire to Eat at Week 12 (p<0.001) and a 43.1% decrease on Eat How Much Food (p<0.001) as illustrated in Table 2.

TABLE 2
Study Results of Participants at Week 0, 4, 8, and 12.

| Measured Parameters | Week 0 | Week 4 | Week 8 | Week 12 |
|---|---|---|---|---|
| Exercise Frequency | 3.3 ± 1.3 | 3.3 ± 0.5 | 2.7 ± 0.8 | 2.4 ± 1.2 * |
| Energy Level | 5.9 ± 1.5 | 6.5 ± 1.3 * | 6.5 ± 1.6 * | 6.5 ± 1.6 * |
| Hunger Level | 6.4 ± 1.6 | 4.0 ± 1.7 *** | 4.2 ± 1.1 *** | 3.7 ± 1.2 |
| Satiety Level | 4.1 ± 1.1 | 6.3 ± 1.3 *** | 6.1 ± 1.2 *** | 7.4 ± 1.0 *** |
| Desire to Eat | 7.2 ± 1.4 | 4.6 ± 1.1 *** | 4.2 ± 1.6 *** | 3.7 ± 2.1 |
| Eat How Much | 5.8 ± 1.0 | 4.2 ± 0.8 | 3.6 ± 0.8 *** | 3.3 ± 1.4 *** |
| Basal Metabolic Rate | 1633.7 ± 278.2 | 1642.1 ± 294.9 | 1634.2 ± 287.9 | 1636.9 ± 288.0 |
| (BMR) (kcal) | | | | |
| Systolic Pressure | 122.8 ± 10.7 | 119.7± 11.8 | 119.3 ± 11.5 | 114.6 ± 8.9 |
| (mmHg) | | | | |
| Diastolic Pressure | 79.7 ± 7.1 | 76.7 ± 6.5 | 75.4 ± 11.2 * | 74.4 ± 8.9 * |
| (mmHg) | | | | |
| Heart Rate (bpm) | 60.1 ± 2.6 | 59.2 ± 8.0 | 56.3 ± 4.9 | 56.4 ± 7.5 |
| Total Cholesterol | 166.4 ± 30.2 | 157.2 ± 29.3 | 143.0 ± 11.8 ** | 141.0 ± 15.3 ** |
| (mg/dL) | | | | |
| Fasting Glucose | 89.8 ± 4.8 | 85.5 ± 7.4 * | 81.3 ± 6.7 *** | 82.0 ± 6.3 |
| (mg/dL) | | | | |
| Insulin (pmol/L) | 43.2 ± 2.9 | 39.4 ± 3.5 *** | 38.3 ± 2.9 * | 36.1 ± 2.4 * |
| HbA1C (%) | 5.3 ± 0.2 | 5.3 ± 0.1 | 5.3 ± 0.1 | 5.1 ± 0.1 |
| GLP-1 (pmol/L) | 6.5 ± 1.1 | 6.8 ± 1.0 *** | 6.9 ± 1.0 *** | 7.3 ± 0.9 *** |
| Body Weight (lbs) | 171.3 ± 24.3 | 168.6 ± 25.2 ** * | 166.1 ± 25.0 * * | 163.4 ± 24.4 |
| Body Mass Index | 27.1 ±.3 | 26.5 ± 1.2 *** | 26.1 ± 1.3 | 25.7 ± 1.4 ** |
| (BMI) (kg/m²) | | | | |
| Body Fat Percentage | 25.2 ± 8.8 | 23.7 ± 8.6 *** | 23.1 ± 8.7 | 21.5 ± 9.2 *: |
| (%) | | | | |
| Fat Mass (FM) (lbs) | 42.3 ± 13.5 | 38.9 ± 12.4 *** | 37.3 ± 12.4 *** | 34.1 ± 13.5 ** |
| Fat Free Mass (FFM) | 129.0 ± 28.6 | 129.8 ± 30.0 | 128.8 ± 29.6 | 129.3 ± 29.5 |
| (lbs) | | | | |
| Waist Hip Ratio | 0.875 ± 0.034 | 0.862 ± 0.027 | 0.860 ± 0.016 * | 0.857 ± 0.018 * |
| (WHR) | | | | |
| Body Fat Index (BFI) | 6.815 ± 2.474 | 6.273 ± 2.298 *** | 6.031 ± 2.304 *** | 5.530 ± 2.440 *** |
| (kg/m²) | | | | |

Results in Table 2 were presented as Mean ± SD.
* indicates 0.01 < p < 0.05;
** indicates 0.001 < p < 0.01;
*** indicates p < 0.001 in comparison to results at Week 0.
Exercise Frequency is defined as follows: How frequently did you exercise this past month? [1-0-5 days | 2-6-10 days | 3-11-15 days | 4-16-20 days | 5-20+ days]. Energy Level, Hunger Level, Satiety Level, Desire to Eat, and Eat How Much are on a scale from 1 to 10, with 1 being the lowest and 10 being the highest.

### Basal Metabolic Rate, Blood Pressures, Heart Rate

There was no significant change in basal metabolic rate (p>0.05) among study participants during the study. Study participants saw a trending decrease in heart rate on average over time during the study, but not statistically significant (Week 12 vs Week 0:6.2% decrease; p>0.05). However, study participants saw significant decreases of blood pressures over time during the study, specifically, a 6.7% decrease of Systolic Pressure on average from Week 0 to Week 12 (p<0.001) and a 6.6% decrease of Diastolic Pressure on average from Week 0 to Week 12 (p<0.05) as illustrated in Table 2.

Blood Biochemical Parameters: Fasting Glucose, Insulin, HbA1c, GLP-1, Total Cholesterol.

Study participants saw a significant decrease of fasting Total Cholesterol over time during the study, specifically an average 15.3% decrease from 166.4 mg/dL at Week 0 to 141.0 mg/dL at Week 12 (p<0.01).

Fasting blood glucose and insulin both saw significant reductions over time (from Week 0 to Week 4, 8 and 12) during the study. Specifically, from Week 0 to Week 12 on average, there was an 8.7% reduction of fasting blood glucose from 89.8 to 82 mg/dL (p<0.001) and a 16.4% reduction of fasting blood insulin from 43.2 to 36.1 pmol/L (p<0.001). Fasting HbA1c levels did not see a significant reduction until Week 12: a 3.8% reduction of fasting HbA1c from 5.33% to 5.13% (p<0.001). Study participants saw a significant improvement of fasting blood GLP-1 over time during the study, specifically a 12.0% improvement from 6.49 pmol/L at Week 0 to 7.27 pmol/L at Week 12 (p<0.0001) as illustrated in Table 2.

### Anthropometric Parameters: Body Weight, Fat Mass (FM), Fat Free Mass (FFM), Body Fat Percentage, Waist Hip Ratio (WHR), Body Mass Index (BMI), Body Fat Index (BFI)

Study participants saw a significant reduction in Body Weight over the 12-week study duration, with an average 4.6% reduction at 7.9 lbs (171.29 vs 163.39 lbs, p<0.001). Interestingly, there was also a significant reduction in body Fat Mass (FM) among study participants, with an average 19.3% reduction at 8.2 lbs (42.26 vs 34.11 lbs, p<0.001). Fat Free Mass (FFM) did not show significant change over the 12-week study duration (p>0.05). Correspondingly, both Body Fat Percentage and Body Fat Index (BFI) of study participants saw significant decreases from Week 0 to Week 12; specifically, a 14.7% decrease of Fat Percentage from 25.24% to 21.24% (p<0.001) and a 18.8% decrease of BFI from 6.81 to 5.53 kg/m² (p<0.001). There was also a significant decrease of Body Mass Index (BMI) for study participants over the 12-week duration: 4.7% decrease from 26.95 to 25.69 kg/m² (p<0.001). Waist Hip Ratio (WHR) also went down from 0.875 at Week 0 to 0.857 at Week 12 (p<0.05) as illustrated in Table 2.

The comprehensive assessment of various parameters throughout the 12-week clinical study provided valuable insights into the efficacy and safety of GB in improving metabolic health and body composition. Each measured parameter reflects specific aspects of metabolic function, and the observed changes can be attributed to the individual and synergistic effects of the active ingredients contained in the study product.

### Hunger, Satiety and Energy Levels

The subjective parameters assessed in this study provide valuable insights into the participants' perceived changes in energy levels, hunger, fullness, and eating behaviors during the 12-week study duration. The observed increase in energy levels, despite a reported decrease in exercise frequency, suggests a potential pro-metabolic effect of GB. This increase in energy could be attributed to the combination of botanical extracts and essential trace mineral chromium, such as green tea extract and ginseng root extract. Green tea extract has been associated with increased energy expenditure, fat oxidation, and thermogenesis due to its high catechin content, while ginseng extract has been shown to improve endurance, reduce fatigue, and enhance physical performance.

The significant reductions in feelings of hunger, desire to eat and eat how much, coupled with increased feelings of fullness, indicates a strong appetite-suppressing effects of the study product. Components such as fenugreek seed extract and white kidney bean extract may play key roles in these effects. Fenugreek has been shown to delay gastric emptying and increase satiety due to its high soluble fiber content, while white kidney bean extract inhibits starch digestion and reduces postprandial glucose spikes, potentially prolonging feelings of fullness.

### Basal Metabolic Rate, Blood Pressures, Heart Rate

While basal metabolic rate remained unchanged throughout the study, significant decreases were observed in both systolic and diastolic blood pressures. This reduction in blood pressure aligns with previous research on the cardiovascular benefits of certain botanical extracts. For instance, turmeric root extract contains curcumin, which exhibits anti-inflammatory properties that may contribute to improved vascular health and reduced blood pressure.

The non-significant decrease in heart rate suggests a trend towards cardiovascular benefits, although further research may be needed to elucidate this effect. Components such as black pepper extract, containing piperine, have been associated with improved cardiovascular health through mechanisms such as enhanced vasodilation and reduced oxidative stress.

### Blood Biochemical Parameters: Fasting Glucose, Insulin, HbA1c, GLP-1, Total Cholesterol

The significant reductions in fasting glucose, insulin, HbA1c, and total cholesterol levels indicate improved glycemic control and lipid metabolism with GB. Several active ingredients may contribute to these effects. For instance, green tea extract has been shown to improve insulin sensitivity and reduce fasting glucose levels. Additionally, fenugreek seed extract has been shown to improve glycemic control and lipid profiles in individuals with type 2 diabetes.

The observed increase in fasting GLP-1 levels further supports the potential of GB to enhance hormone signaling and glucose homeostasis and help glucose control. GLP-1 is an incretin hormone involved in glucose-dependent insulin secretion and appetite regulation. Components contained in GB have been shown to increase GLP-1 production via multiple pathways.

It is also interesting that GB significantly reduced fasting glucose and insulin levels, and increased GLP-1 level as early as Week 4 and through Week 12 yet did not significantly reduce HbA1c level until Week 12. These outcomes are consistent with the fact that HbA1c level reflects average fasting glucose over the past 3 months.

Worthy to note that the significant reduction of total cholesterol along with blood pressures, indicated a strong cardioprotective effect of the study product. High cholesterol and high blood pressures are the top risk factors for cardiovascular diseases in the USA and globally. By reducing the top 2 risk factors, the study product can be an effective approach to help reduce the risk of people getting cardiovascular diseases such as heart attacks, strokes.

### Anthropometric Parameters: Fat Loss, Weight Loss and Body Composition

The significant reductions in body weight, fat mass, body fat percentage, BFI, and BMI highlight the potential of the study product for fat loss and weight management. Active ingredients such as gardenia fructus extract and turmeric root extract have been associated with anti-obesity effects through mechanisms such as inhibition of adipogenesis and modulation of lipid metabolism. Similarly, white kidney bean extract may contribute to weight loss by inhibiting starch digestion and reducing caloric absorption.

The observed improvements in waist-hip ratio suggest a favorable redistribution of body fat, potentially further reducing cardiovascular risk. Components such as black pepper extract and ginseng root extract have been associated with reductions in visceral fat and improvements in body composition.

### Brief Comparison of GB to Rybelsus (Semaglutide)

Different GLP-1 RA drugs showed variable outcomes in reducing blood glucose levels, HbA1c levels, body weight and fat mass in different study settings. For this brief comparison, the study of Rybelsus which is an oral format of GLP-1RA drug Semaglutide, is used.

The observed average weight loss among study participants in the present study is about 7.9 lbs over the 12-week study period. This amount does not seem a lot, however, is comparable to and even better than weight loss outcome among study participants who took Rebylsus (high dose 14 mg), who on average lost 8.4 lbs over the course of 6-months. Daily take of 14 mg of Rebylsus for 6-months among obese patients also significantly reduced blood HbA1c level. The present study showed daily take of GB for only 12 weeks already significantly reduced blood HbA1c levels.

Of note, the present study did not show significant change in muscle mass among study participants. This can be a significant advantage compared to GLP-1 RA drugs, which are often associated with muscle loss. Maintaining or increasing muscle mass is especially important for healthy weight management, as an equal amount of muscle mass tends to burn more calories than the same amount of fat mass. Another notable difference between GB and GLP-1 RA drugs such as Rybelsus is that all these drugs come with a long list of side effects, ranging from common ones such as digestive blockage to severe ones including thyroid cancers, while no adverse effects were found or reported for GB.

### Potential Mechanism of Action

Though further and more specific investigations are needed to reveal the detailed mechanisms of action of GB, the observed outcomes from the present study suggested that all active components individually and synergistically increased the secretion of endogenous GLP-1 hormone and employed multiple pathways to metabolic modulation. Elevated GLP-1 levels stimulate insulin release, which helps reduce blood glucose levels. Improved endogenous secretion and regulation of GLP-1 positively impacted insulin sensitivity. Elevated GLP-1 levels also suppress appetite and slow gastric emptying and thus help body weight loss and body fat loss. Elevated GLP-1 level itself and the resulting fat loss, healthier cholesterol and blood pressure levels further contribute to better overall metabolic health and cardiovascular health.

### CONCLUSIONS

In this example, GB was formulated containing a combination of botanical extracts. The data demonstrated that GB exerts beneficial effects on subjective parameters, blood pressures, biochemical parameters, and anthropometric parameters associated with metabolic health, glucose control and weight management. The results showed that GB significantly reduces the fasting blood glucose and insulin levels, and specifically lowered body weight and fat mass among study participants. The effective outcomes were observed and are consistent with the known pharmacological properties of the active ingredients. Moreover, GB contributed to improvements in energy metabolism, appetite regulation, glycemic control, lipid metabolism, and body composition. Furthermore, a randomized placebo-controlled double-blinded study with a bigger number of study participants should be carried out to confirm the efficacy of GB. More research should be performed to elucidate the underlying mechanisms and clinically applied in management of metabolic disorders.

The words “example” or “implementation” are used herein to illustrate an example, instance, or illustration, and is not necessarily to be construed as preferred or advantageous over other aspects or designs. The term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an implementation” or “one implementation” throughout is not intended to mean the same embodiment or implementation unless described as such.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A mammal glucagon-like peptide 1 modulating composition comprising:
green tea leaf extract; gardenia fructus fruit powder; turmeric root extract; black pepper extract; fenugreek seed extract; ginseng root extract; and white kidney bean extract,
wherein the green tea leaf extract comprises 30% or more of epigallocatechin gallate by dry weight;
wherein the turmeric root extract comprises 95% or more of curcumin by dry weight;
wherein the black pepper extract comprises 95% or more of piperine by dry weight;
wherein the ginseng root extract comprises 25% or more of ginsenosides by dry weight; and
wherein the fenugreek seed extract and the white kidney bean extract were enriched in a 4:1 ratio.

2. The mammal glucagon-like peptide 1 modulating composition of claim 1, further comprising:
one or more selected from the group consisting of Citrus aurantium extract, Gentiana scabra extract, Bupleurum falcatum extract, Anemarrhena asphodeloides extract, Plasmodium falciparum extract, Sophorae flos extract, Alismatis rhizome extract, Nelumbinis folium extract, Cassiae semen extract, Poria cocos extract, Zea mays extract, α-linolenic acid, and Hoodia gordonii extract.

3. The mammal glucagon-like peptide 1 modulating composition of claim 1, further comprising:
one or more selected from the group consisting of α-linolenic acid, Bupleurum falcatum extract, Gentiana scabra extract, Hoodia gordonii extract, and Zea mays extract.

4. The mammal glucagon-like peptide 1 modulating composition of claim 1, further comprising chromium, wherein the chromium is chromium nicotinate.

5. The mammal glucagon-like peptide 1 modulating composition of claim 1, further comprising berberine, wherein the berberine is from Berberis Aristata bark extract.

6. The mammal glucagon-like peptide 1 modulating composition of claim 1, wherein the mammal glucagon-like peptide 1 modulating composition is decaffeinated.

7. The mammal glucagon-like peptide 1 modulating composition of claim 1, wherein the amount of each of the components corresponds to a ratio of:
0.1-1 green tea leaf extract; 0.1-1 gardenia fructus fruit powder; 0.04-1 turmeric root extract; 0.004-1 black pepper extract; 0.04-1 fenugreek seed extract; 0.04-1 ginseng root extract; and 0.04-1 white kidney bean extract.

8. The mammal glucagon-like peptide 1 modulating composition of claim 1, further comprising:
one or more selected from the group consisting of vitamin C, vitamin D, calcium, magnesium, phosphate, pyruvate, NADH, nitrates, and a mixture thereof.

9. The mammal glucagon-like peptide 1 modulating composition of claim 1, wherein the composition is administered in solid, semi-solid, liquid, semi-liquid, powder, suspension, emulsion, solution, ready-to-drink beverage, gel, bar, pill, tablet or capsule form.

10. A method of modulating mammal glucagon-like peptide 1 secretion comprising administering to a mammal the mammal glucagon-like peptide 1 modulating composition of claim 1.

11. The method of claim 10 further comprising daily oral administration of the mammal glucagon-like peptide 1 modulating composition of claim 1.

12. The method of claim 10, wherein the mammal glucagon-like peptide 1 modulating composition of claim 1 is in solid, semi-solid, liquid, semi-liquid, powder, suspension, emulsion, solution, ready-to-drink beverage, gel, bar, pill, tablet or capsule form.

13. A method of stimulating body weight loss in a mammal comprising administering to the mammal the mammal glucagon-like peptide 1 modulating composition of claim 1.

14. The method of claim 13 further comprising daily oral administration of the mammal glucagon-like peptide 1 modulating composition of claim 1.

15. The method of claim 13, wherein the mammal glucagon-like peptide 1 modulating composition of claim 1 is in solid, semi-solid, liquid, semi-liquid, powder, suspension, emulsion, solution, ready-to-drink beverage, gel, bar, pill, tablet or capsule form.
